(19) United States
(12) Plant Patent Application Publication
(10) Pub. No.: US 2015/0020249 P1
(43) Pub. Date: Jan. 15, 2015

(54) Strawberry plant named 'Dream'

(50) ;

(71) Applicant: Alexandre Pierron-Darbonne, Navarra (ES)

(72) Inventor: Alexandre Pierron-Darbonne, Navarra (ES)

(21) Appl. No.: 13/987,148

(22) Filed: Jul. 3, 2013

(51) Int. Cl. A01H 5/00

(52) U.S. Cl. PLT/208

(57) ABSTRACT

A new and distinct strawberry variety, Fragaria×ananassa, cv. “Dream” is characterized by a red to dark red fruit color, a large fruit size, an open density, and a medium vigor.

### LATIN NAME OF THE GENUS AND SPECIES CLAIMED

Fragaria×ananassa

### VARIETY DENOMINATION

‘Dream’

### BACKGROUND AND SUMMARY OF THE INVENTION

The present invention relates to a new and distinct strawberry variety. The varietal denomination of the new variety is “Dream”. The new variety was designated by the breeder as “Dream (DA50)”. The new variety of strawberry was created in a breeding program by crossing two parents in 2007 in Le Barp, France; in particular, by crossing as seed parent an undistributed strawberry parent designated “31.14.04” (unpatented) and as pollen parent an strawberry parent designated “01.12.52” (unpatented). Seed parent and pollen parent are selections from the breeder's program and have not been commercialized.

The resulting seedling of the new variety was grown and asexually propagated by runners in Le Barp, France 0.7° W., 44° , 50 meters elevation. Clones of the new variety were further asexually propagated and extensively tested. This propagation and testing has demonstrated that the combination of traits disclosed herein which characterize the new variety are fixed and retained true to type through successive generations of asexual reproduction.

Among the characteristics which appear to distinguish the new variety from its closest variety of which I am aware, “Clery,” is a combination of traits which include: serrate terminal leaflet incisions in the new variety as compared to crenate for “Clery.” The new variety also has a darker red to dark red fruit color, as compared to red for “Clery.” The luminosity for the fruit of the new variety at 460 nm observed on May 7 was 56.7, as compared to 43.5 for “Clery.” The new variety also has a large fruit size as compared to medium for “Clery.” The average fruit mass (g/fruit) observed for “Dream” on May 7 was 23.5 g, compared to 18.9 g for Clery.

Characteristics which appear to distinguish the new variety from the variety “Darselect,” as characterized in U.S Plant Pat. No. 10,402, at least include that the new variety has an open density and a medium vigor.

### BRIEF DESCRIPTION OF PHOTOGRAPHS

The accompanying photographs show typical specimens of the new variety, designated “Dream” or “DA 50” in the illustrations, including fruit, foliage and flower, in color as nearly true as it is reasonably possible to make in color illustrations of this character.

FIG. 1 shows several plants of the new variety “Dream.”

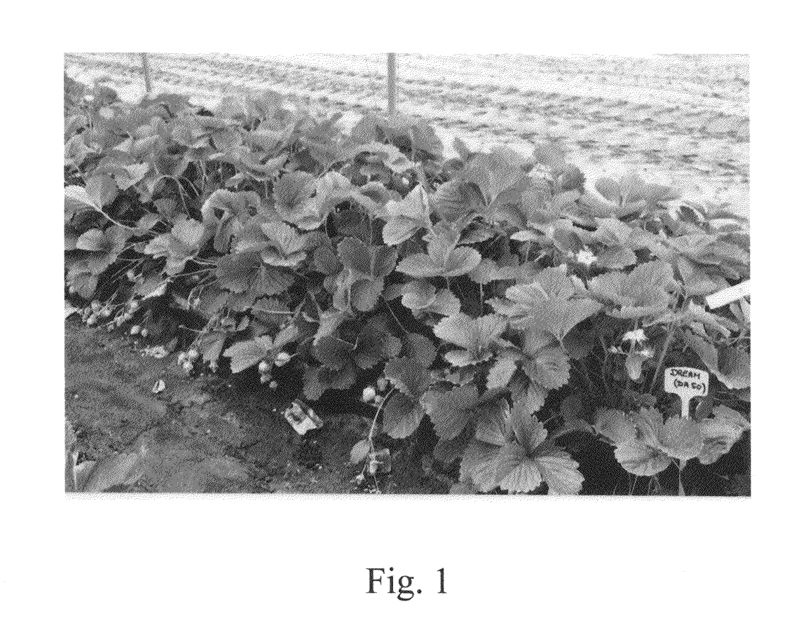

FIG. 2 shows several plants of the closest variety, “Clery.”

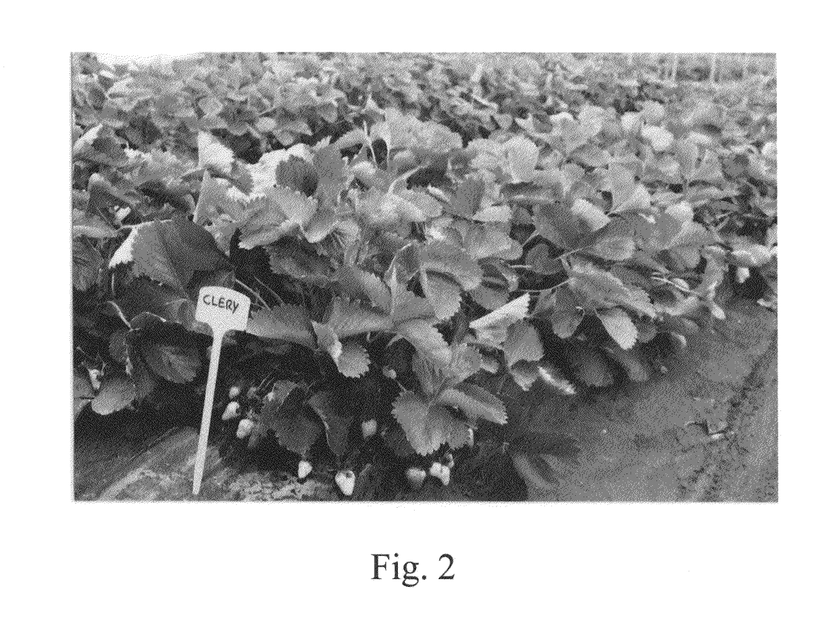

FIG. 3 shows several plants of the new variety of “Dream” with several red colored and conical shaped fruits.

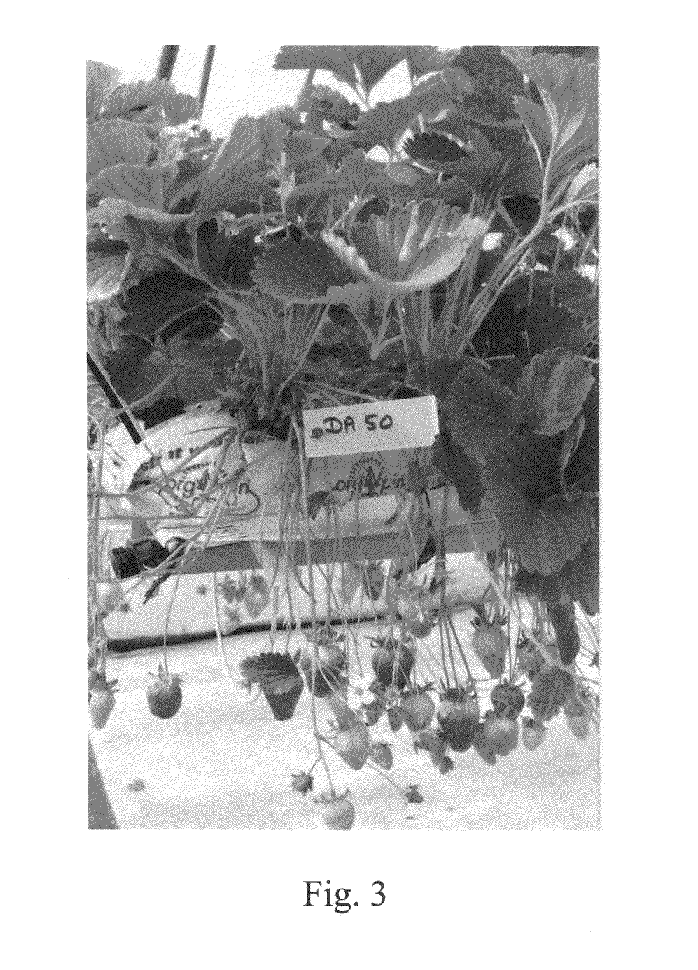

FIG. 4 shows the upper side of a terminal leaflet of the new variety “Dream.” A medium green color, and serrate shapes of incisions at the margins of the terminal leaflets can be seen.

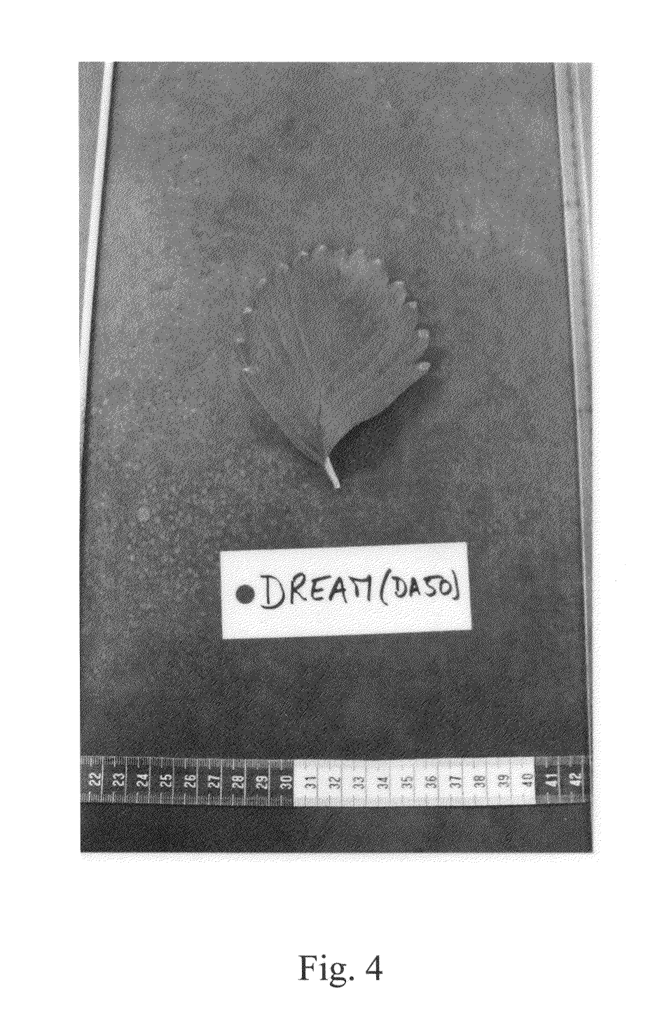

FIG. 5 shows the upper side of a terminal leaflet of the variety “Clery.” Crenate shapes of incisions at the margins of the terminal leaflets can be seen.

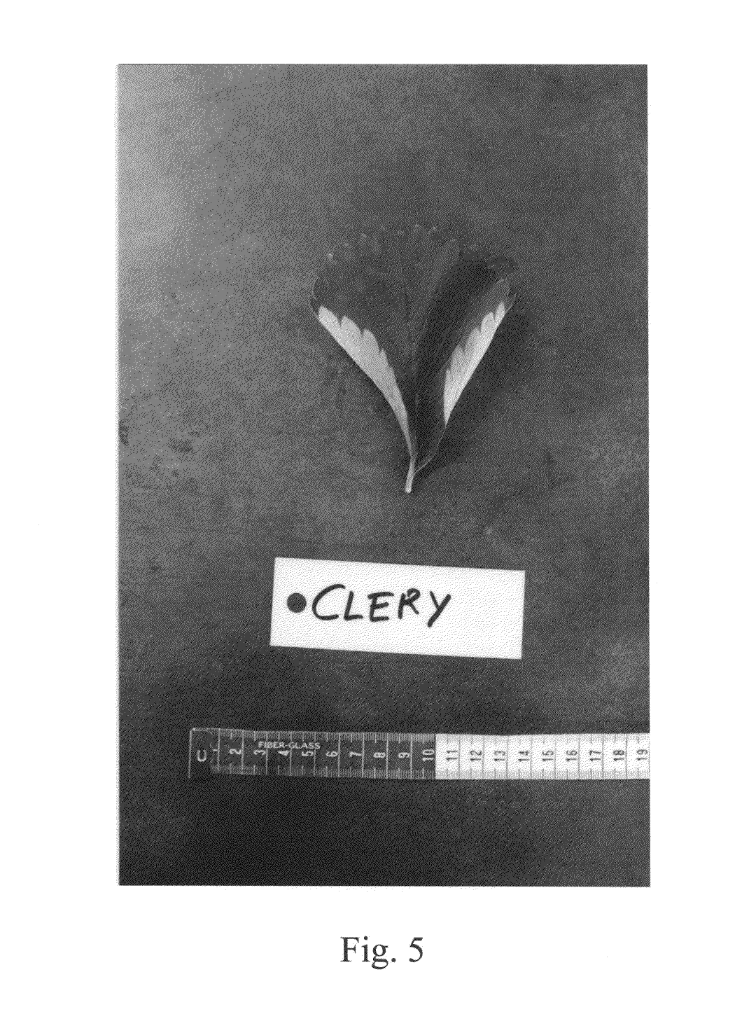

FIG. 6 shows the upper side of a complete leaf of the new variety “Dream.” A slightly concave cross-section can be seen.

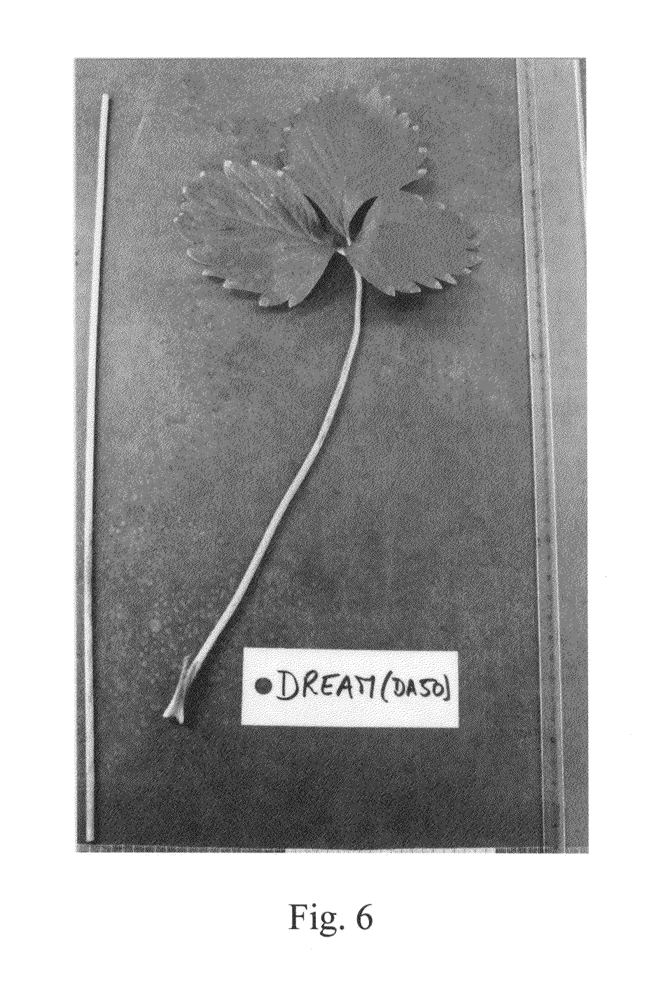

FIG. 7 shows the lower side of a complete leaf of the new variety, “Dream.”

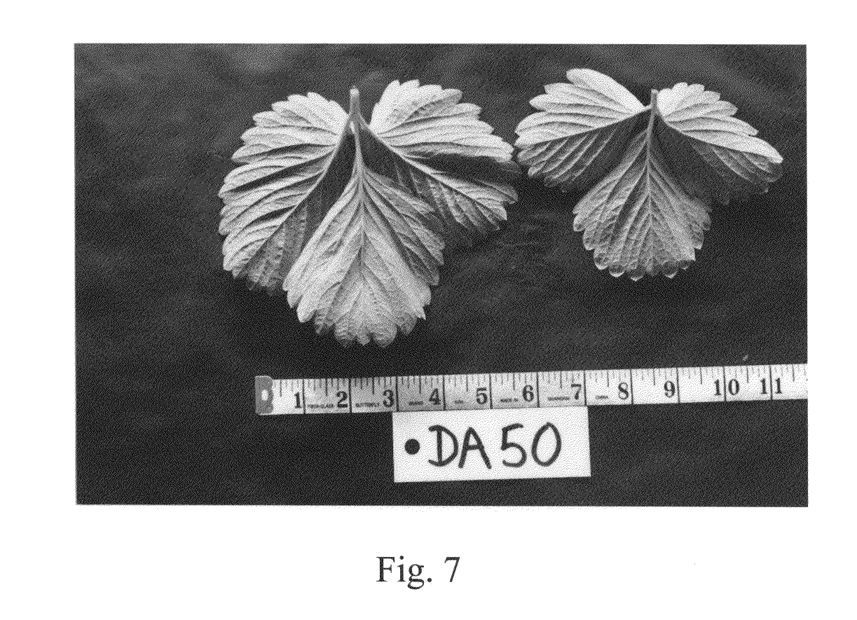

FIG. 8 shows the stipule and petiole of the new variety, “Dream.”

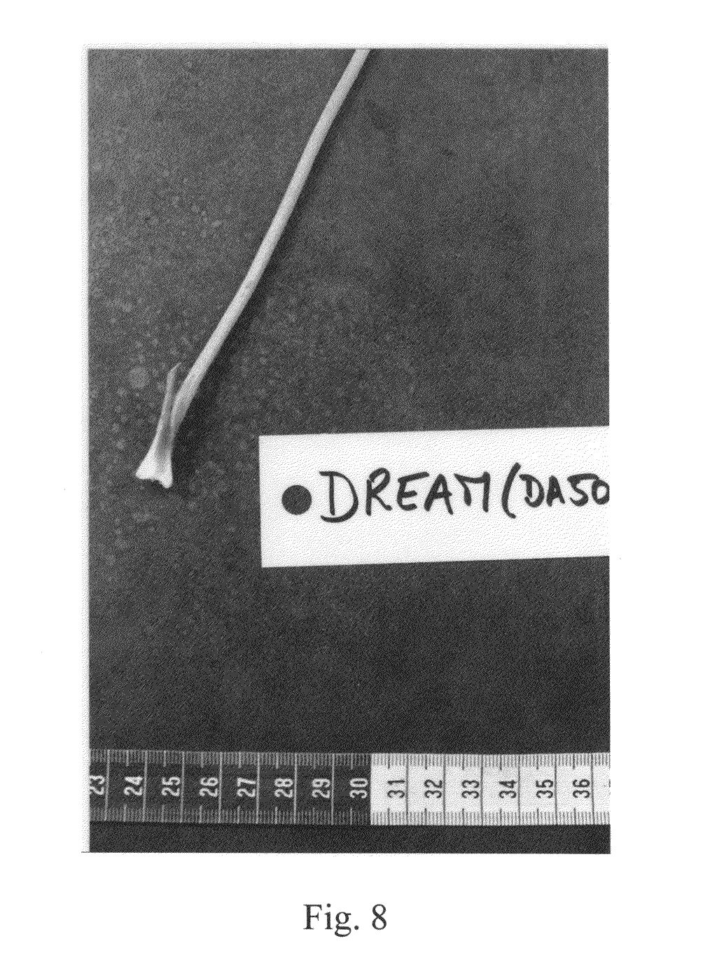

FIG. 9 shows the stipule and petiole of the variety “Clery.”

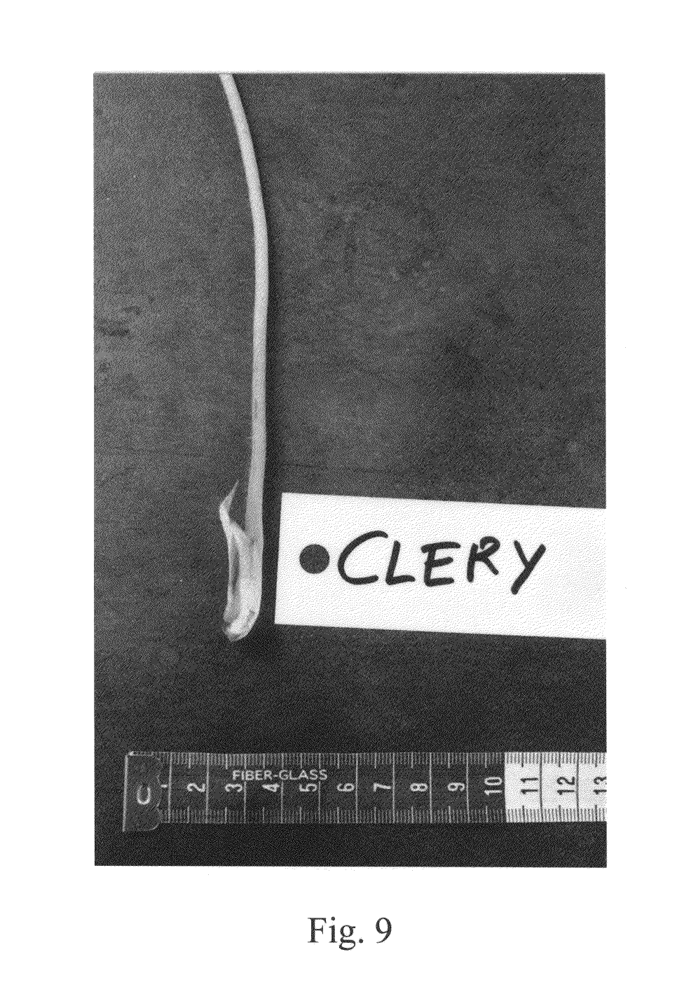

FIG. 10 shows the flower of the new variety, “Dream.”

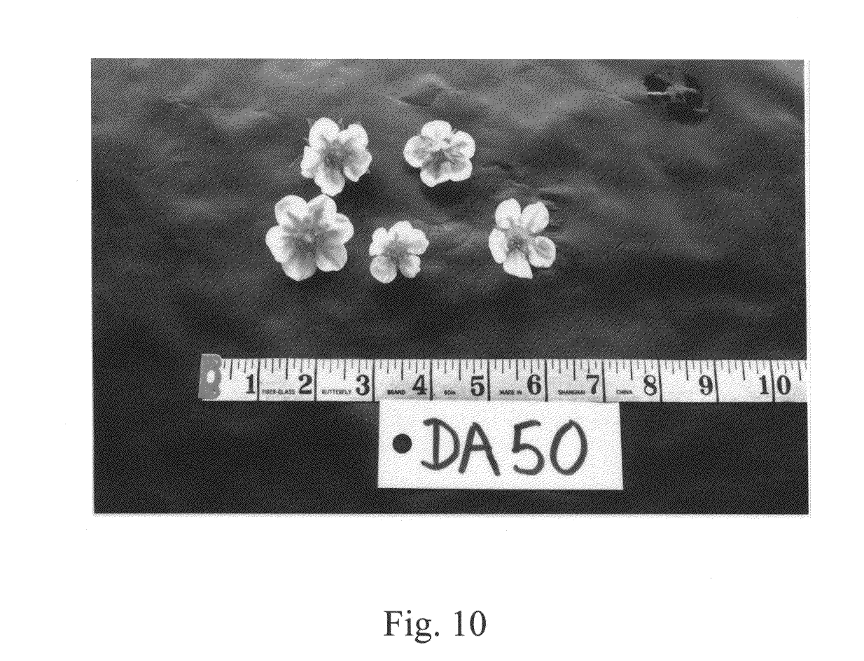

FIG. 11 shows typical whole fruit of the new variety “Dream,” illustrating the typical conical shape, red color, and strong glossiness.

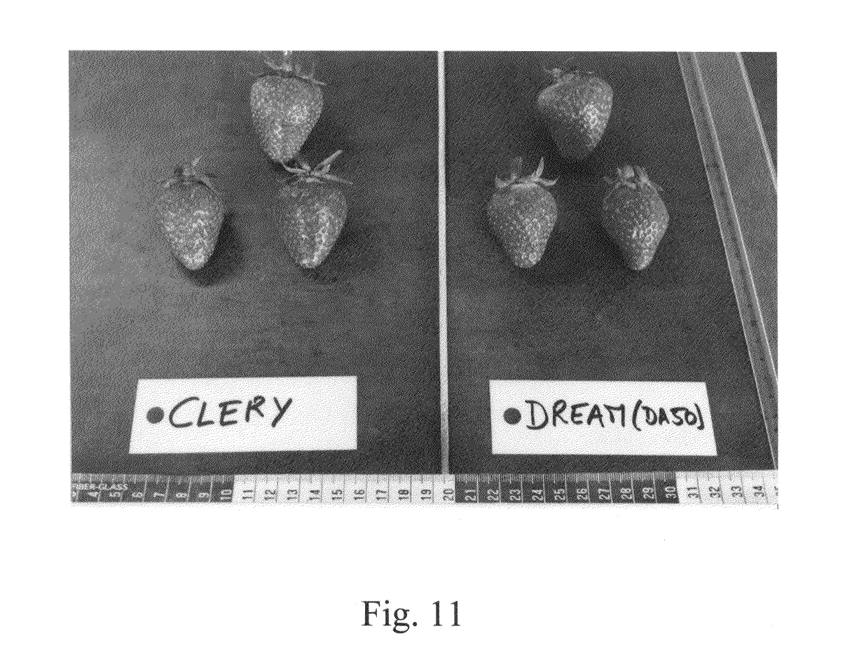

FIG. 12 shows a typical sliced section of the fruit of the new variety “Dream,” illustrating the typical flesh coloration of about light red and a very weakly expressed hollow center.

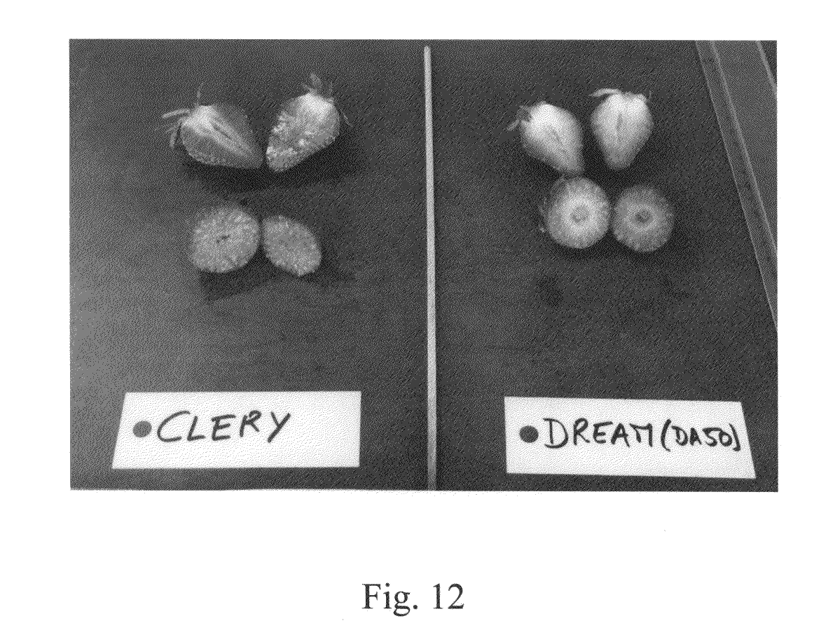

### DESCRIPTION OF THE NEW VARIETY

Throughout this specification, color names beginning with a small letter signify that the name of that color, as used in common speech is aptly descriptive. Color names beginning with a capital letter designate values based upon the R.H.S. Colour Chart published by the Royal Horticultural Society, London, England, 1995. The color descriptions and other phenotypical descriptions may deviate from the stated values and descriptions depending upon variation in environmental, seasonal, climatic and cultural conditions.

The following detailed description of the new variety is based upon observations taken of plants and fruits grown in Le Barp, France 0.7° W., 44°, 50 meters elevation.

The new variety is principally propagated by way of runners. Although propagation by runners is presently preferred, other known methods of propagating strawberry plants may be used. Strawberries root well after transplanting.

The term “blistering” used herein refers to the texture or rugosity or surface ondulation inherent to leaves and is generally a constant characteristic.

Table 1 shows the Weight (g/Fruit) on May 7 for the new variety “Dream,” and two varieties, “Clery” and “Darselect.”

TABLE 1

| WEIGHT (g/fruit)ⁱ | May 7 |
|---|---|
| Dream | 23.5 |
| Clery | 18.9 |
| Darselect | 24.5 |

ⁱWEIGHT is shown as the average weight per fruit in First Quality Fruits.

Table 2 shows a comparison of the fruit analysis on May 7 between the new variety “Dream,” and two varieties, “Clery” and “Darselect.”

TABLE 2

| | DREAM | CLERY | DARSELECT |
|---|---|---|---|
| Humidity & Volatile Matter (%) | 92.6 | 91.5 | 91.5 |
| Dry Matter (%) | 7.4 | 8.5 | 8.5 |
| pH (to 20°) | 3.9 | 3.8 | 3.8 |
| Acidity as Anhydride Citric (%) | 0.5 | 0.6 | 0.6 |
| Soluble solids (°Brix) | 7.4 | 7.9 | 7.8 |
| Maturity Index | 14.8 | 13.2 | 13.0 |
| Dominant Tonality (nm) | 490 | 495 | 490 |
| Luminosity: Transmittance to 460 nm | 56.7 | 43.5 | 51.2 |

B. Dry Matter: It is the weight of the residual left from the trituration of the fruit after the drying process at a temperature of 103° C. +2° C. until reaching constant weight.

$$\% Dry Matter = \frac{Weight Dry Matter}{Weight Fresh Matter} \times 100$$

C. Humidity & Volatile Matter: Represents the content in volatile matters and water of the fruits.

(%) Humidity & Volatile Matter=100−% Dry Matter
D. Maturity Index: Relation between Soluble solids and Acidity as Anhydride Citric.

$$Maturity Index = \frac{Soluble solids}{Acidity as Anhydride Citric}$$

### DETAILED DESCRIPTION OF THE NEW VARIETY

Plant:
   Growth habit.—Semi upright.
   Habit.—Flat globose.
   Density.—Open.
   Vigor.—Medium.
Leaf:
   Size.—Medium.
   Color of upperside.—Medium green.
   Cross section.—Slightly concave.
   Blistering.—Weak.
   Glossiness.—Medium.
Terminal leaflet:
   Length/width ratio.—As long as broad.
   Terminal leaflet shape of base.—Obtuse.
   Shape of incisions at margin.—Serrate.
Petiole:
   Attitude of hairs.—Slightly outwards.
Stipule:
   Size.—Medium.
   Anthocyanin coloration.—Weak.
Stolons:
   Number.—Medium.
   Antocyanin coloration.—Weak.
   Pubescence.—Weak.
Inflorescence:
   Position relative to foliage.—Above.
Flower:
   Size.—Medium.
   Size of calyx relative to corolla.—Smaller.
   Primary flower relative position of petals.—Touching.
Petal:
   Length/width ratio.—Much broader than long.
   Color.—White.
Fruit:
   Ratio of length/maximum width.—Slightly longer than broad.
   Size.—Large.
   Fruit Shape.—Conical.
   Difference in shapes between primary and secondary fruits.—Slight.
   Band without achenes.—Absent or very narrow.
   Unevenness of surface.—Weak.
   Color.—Red.
   Evenness of color.—Slightly uneven.
   Glossiness.—Strong.
   Insertion of achenes.—Below surface.
   Insertion of calyx.—Level with fruit.
   Attitude of the calyx segments.—Reflexed.
   Size of the calyx in relation to fruit diameter.—Same size.
   Adherence of calyx.—Strong.
   Firmness.—Firm.
   Color of flesh.—Light red.
   Hollow center.—Absent or very weakly expressed.
   Distribution of red colour of flesh.—Only marginal.
   Time of flowering.—Very early.
   Time of ripening.—Very early.
   Type of bearing.—Partially remontant.
Disease resistance: No particular sensitivity to any disease or parasite has been observed for “Dream”.

What is claimed is:

1. A new and distinct strawberry plant of the variety substantially as shown and described.
